(19) United States
(12) Patent Application Publication
(10) Pub. No.: US 2012/0059068 A1
(43) Pub. Date: Mar. 8, 2012

(54) Composition Based On Zeolite And Usage As Lick Stone

(75) Inventors: Stephan Hugo Frei, Hume Park 2 (SG); Alfred Ernst Gehring, Stans (CH)

(73) Assignee: TRIPLE-S PTE LTD, (SG)

(21) Appl. No.: 13/320,896

(22) PCT Filed: Nov. 5, 2009
(86) PCT No.: PCT/SG2009/000407

§ 371 Date: Nov. 16, 2011

(30) Foreign Application Priority Data
May 25, 2009 (CH) 00806/09

(51) Int. Cl. A23K 1/175; A23P 1/10; A61K 47/02

(52) U.S. Cl. 514/770; 426/71; 426/271

(57) ABSTRACT

The invention lies in the field of environmental technology and relates to a compound, in particular for the use, in particular as metabolically inert gas absorber for ruminants on the basis of zeolite, salt and a binding agent. It furthermore relates to a licking element with such a composition, a process for the production of such a licking element as well as its application. The use of zeolite relieves the organism of toxic substances, such as heavy metals and toxins. Clinoptilolite is a dietetically working ballast material, which stabilises the intestinal activities without depriving the body of nutrients such as vitamins and trace elements. Thus it is possible to achieve a reduction of the gas expulsion from the digestive tract. The licking element can be set up in the open, so that even free-ranging animals that belong to a farm can be reached in an easy manner.

### FIELD AND BACKGROUND OF THE INVENTION

The invention belongs to the field of environment technology and concerns a compound, in particular for usage as metabolically inert gas absorber for ruminants, with the characteristics of claim 1, a licking element including such a compound in accordance with claim 8, a process for the production of a licking element in accordance with claim 11, and a process for the production of the compound in accordance with claim 15.

The production of greenhouse gases is seen as one of the main reasons for changes in the climate. The main sources of greenhouse gases are the industry, cars and agriculture. Due to, inter alia, the methane discharge of ruminants caused by their digestion, agriculture is the largest producer of CO₂ equivalents.

The following approaches for the reduction of the methane production by ruminants have been proposed or have already been tried out:
Modification of the DNA of the animals in such way that inherently less greenhouse gases are produced: That approach would probably technically only be feasible at great cost and lead to resistance in the population (gene modification).
Vaccination of the animals: That approach would probably only be feasible with regard to animals kept in stables, but only at great cost and additional expenditures (veterinarian, vaccine).
Use of a garlic extract as feed additive: With regard to this, changes in the quality of the milk and meat products are expected (smell, texture).
The admixture of fish oil to the feed has also been tried. This is probably less suitable for large-scale use considering that fish stocks already are under considerable strain.

Therefore the invention has the task to provide efficient and inexpensive means for the reduction of the methane production by ruminants, which can be deployed over large areas, are not limited to farm animals kept in stables and do not affect the quality of the agricultural products.

### SUMMARY OF THE INVENTION

This task is addressed by a compound, which can be used in particular as metabolically inert gas absorber, with the characteristics in accordance with claim 1, by a licking element on the basis of that compound in accordance with claim 8 and by a process for the production of such licking element in accordance with claim 11. The present invention also relates to a process for producing such a compound in accordance with claim 15. Advantageous further developments are illustrated in the dependent claims, the description and the illustrations.

The compound in accordance with the invention, which can be deployed in particular as metabolically inert gas absorber for ruminants, comprises 25-97% by weight of zeolite, 1-20% by weight of salt, in particular a mixture of saline salt and sea salt as well as 1-15% by weight of a binding agent approved for foodstuffs. In addition, the compound may contain further components, for example minerals that are absorbable and/or trace elements for strengthening the immune system and the gastrointestinal flora or pharmaceuticals for the purpose of therapy or prophylaxis, essential oils (e.g. 0.5-5% by weight), natural dyes (e.g. 0.1-5.0% by weight) for the purpose of optical differentiation.

A licking element for ruminants in accordance with the invention comprises at least a moulded part with such a compound in a compact form factor.

The process in accordance with the invention for the production of such a licking element comprises at least the following steps:
Mixing the binding agent with water;
Adding the zeolite, the salt and the other application-specific additives;
Stirring until a homogenous mass has been achieved;
Filling of the mass into a form;
Compression;
Hardening for the creation of the moulded part.

### BRIEF DESCRIPTION OF THE DRAWINGS

An embodiment of the invention will now be described, by way of example only, with reference to the accompany drawings in which:

FIG. 1 shows components of a support for a licking element; and

FIG. 2 shows steps for the installation of the licking element at the place of installation.

### DETAILED DESCRIPTION OF THE PREFERRED EMBODIMENT

The invention is based on the insight that the ion exchanging and adsorption characteristics of zeolites can be utilised in a targeted manner to bind undesirable gases, which are produced in the digestive systems of ruminants and that otherwise would escape via the breath or the intestines, in such way that the corresponding substances are now available to the animal—proportionally to the effectiveness of the zeolite—as a source of energy due to the (catalytic) function and are not excreted as a loss together with the faeces. The compound is absorbed by the animals and in the process adjusts or modifies the digestion naturally. However, the main component zeolite is digestion-neutral, i.e. it is not absorbed by the body. Therefore it is not a feed in the conventional sense of the word, but is rather to be compared with that of a catalyst.

The zeolite starts to work in the first stomach and continues in the subsequent digestive organs, in particular a) in the entire digestive tract, where the proteins are broken down, and especially b) in the rectum, where usually further decomposition and gas formation takes place. Especially methane (CH₄) and ammonia (NH₄⁺) are bound in the present case and thus fed to the animal as energy by way of a bacterial effect, instead of being discharged either by eructation or with the faeces. A further effect is the selective binding of harmful substances, environmental toxins etc. and thus the relief of the entire organism, especially of the liver and the kidneys.

First of all, the products is meant to be used as a consumable preparation and is not applied to the excrement, even though these are significantly changed in consequence of the passing of the product through the digestive tract. Preferred is the application in a solid, compact form as a licking element, which is voluntarily licked by the animal (attracted by the salt contained in it), so that small quantities of the preparation are consumed independently of the normal feeding process. The application as an additive in the form of a powder or granulate, which is mixed with the normal feed and is consumed by the animal in that way, is also conceivable.

Zeolites are crystalline aluminium silicates that exist in numerous variations in nature, but that can also be produced synthetically. The composition of zeolites is: M_{x/n}[(AlO₂)ₓ(SiO₂)_{y}]⁻zH₂O (with n=charge of M, mostly 1, 2). Zeolites have a regular arrangement of hollow spaces and channels, into which water and low-molecular substances can be absorbed and can be released again upon heating, without the crystalline structure of the zeolite being destroyed by that process. So far zeolites are used as catalysts for chemical processes, as materials for the separation of chemical substances or as water softener in washing detergents. In the field of animal feeds they are used in very low concentrations as an integral part of the feed, in order to bind toxic substances. In the foodstuff industry zeolites are used for preventing the sticking together of pourable substances.

The molecular structure of zeolite makes an optimal ion exchange sequence possible with regard to CH₄ and NH₄⁺. Water molecules can be bound in two ways in the pores and hollow spaces of zeolite, i.e. the water in the structural molecules (SiO_{4/4} and AlO_{4/5}) as well as the water, which forms the hydrate envelope of the exchangeable cations. Ions can be adsorbed by way of contact with the zeolite in a water solution. The specific surface (size, geometry and energy character) of the zeolite influences the ion adsorption. In difference to the absorption, the adsorption particles do not penetrate into the crystalline structure of the adsorbent, but are only captured by way of electrostatic on the surface. Due to the energetic and crystallographic difference, the surface of the zeolite is defined as heteropolar and heterogeneous. In the event that the adsorbent—due to the ion exchanging mechanism—is saturated with the adsorbate, salts are physically adsorbed on the surface, e.g. by way of van-der-Waals binding. This results in mineral-specific ion exchange and adsorption characteristics of zeolite.

The ion exchange sequence of zeolite is very specific: NH₄⁺<<K<<Na<Mg<Ca. CH₄ is bound to the same extent as NH₄⁺. The bound nitrogen is virtually completely decomposed by the abundantly available strains of microbes.

This invention is using a substance with a comparatively high weight share of 25-97% by weight of zeolite, preferably 50-90% by weight, especially preferred approx. 80% by weight. The animal is to get the zeolite voluntarily, so that the zeolite may have the desired effect in the digestive tract. For this reason the compound in accordance with the invention also contains a share of 1-20% by weight of (preferably 3-15% by weight) salt, in particular NaCl (pure saline salt, pure sea salt or a mixture thereof), which also attracts free-ranging farm animals, that are not fed in a controlled manner. By way of the binding agent with a share of approx. 3-15% by weight, typically approx. 10% by weight, the salt forms a compound with the small-grained zeolite, so that both components are taken up together.

Preferred is the administration of the compound in the form of a licking element (licking stone, licking mass). However, the compound may also have the form of a powder or granulate that is added to the feed. The advantage of a compact licking element in comparison with a powdery/granulate-based admixture to the feed is that it can be placed anywhere without restrictions. In that way the quantity taken up can be adjusted to the animal, if the licking habits of the animal are known. This has several advantages: On the one hand it is economically advantageous, if a sufficient but not excessive quantity is taken up. In that the licking elements are not worn out or used up prematurely, which reduces the work involved in filling them up again. In contrast to a powder, a solid licking element is also not uncontrollably bloated, compacted or thinned and washed away by environmental influences such as rain. And it can be used in stables or limited runs as well. In the case of an unlimited run, the licking element is placed at the water holes used by the animals. The licking element consists of the aforementioned compound, which is formed into a moulded part of a suitable shape, e.g. a disk, a rod or a block. It can be affixed to a support, which is anchored in the ground or is placed on top of such a support.

In principle the compound can be used for all ruminants, e.g. cattle, sheep, goats, giraffes, bison, yaks, water buffalos, deer, does, camels, alpacas, lamas, gnus, antelopes and such, with focus on the agriculturally relevant species (cattle, sheep, goats, lamas). The size and form of the licking stone can be adjusted to the biological species.

The zeolite preferably has a grain size in the range of 10-150 μm. The size of the pores and channels respectively of the zeolite preferably fall in the range of 0.3 nm to 5 nm (3 to 50 Angstrom), in particular 1-4 nm.

The zeolite component of the compound preferably contains Clinoptilolite or consists exclusively of Clinoptilolite, a zeolite of the Heulandite group, which has proven to be particularly suitable.

Possible binding agents are all sweetener-free substances from E400 to E466 that are approved for foodstuffs as well as modified starches of the groups E1404 to E1451. Grain-based products, such as rye-, wheat- and rice-bran have good binding characteristics. Mixtures of the listed binding agents can be used as well. Substances containing sweeteners, in particular substances containing sugar, must not be used due to the risk of caries in the case of long-term use.

An integrated, biodegradable protection layer, for example a starch-based film, ensures that the licking element does not prematurely decompose when exposed to environmental influences. Such a protective layer, which is preferably applied to the top of the licking element, is used in particular in locations with increased precipitation, e.g. the tropics. Preferably, that layer will meet the European standard for biodegradability EN 13432.

With regard to its form and size the licking element should be adjusted to the target group of animals, e.g. be smaller for sheep than for cattle. The form has been chosen in such way that the administration as well as the consumption by the animal is simplified. For example, the licking element has the form of a disk with a hole in the centre, into which a support can be inserted, preferably secured against rotating. A typical diameter of such an element lies in the range of 30 cm; the central hole has dimensions of, for example, 4×6 cm. The central hole should not be too large, in order to maintain an optimum utilisation of space during transport.

The mass of the licking element is adjusted to the type of application: For free-ranging animals it may have a mass of 10-20 kg, so that it doesn't have to be replenished so frequently. Preferably, the mass is adjusted to consumption: Depending on the animal, consumption is different due to different licking intensities, different dosages might be necessary as well, in order to achieve the desired effect.

Preferably, the hardness of the licking element is also adjusted to the target animal group. For example, a licking element for cattle is harder than one for sheep. The composition can also be adjusted to the target animal group and its particular needs with regard to nutrition. The composition can be adjusted, in order to provide the animals with additional substances in controlled dosages in a targeted manner, e.g. trace elements, minerals, vitamins, pharmaceuticals.

The licking element is produced preferably as follows:

1. The binding agent, for example rice bran, with 1-30% by weight of the target mass is mixed with de-ionised water and the mixture is then heated to approx. 60-80° C. Stirring until the bran starts to dissolve.

2. Further adding of de-ionised water while at the same time adding the remaining ingredients and mixing of the same, i.e. at least zeolite and salt, until a homogenous mass has been created.

3. In accordance with Item 1 to 2 letting the mass stand for a certain period of time, which depends on the mixture and thus the target species, allowing the escape of possible gas bubbles. Vibration for accelerating the discharge of the gas bubbles is not desirable, while a vacuum-assisted out-gassing may be useful.

4. Pouring of the mass into a suitable mould, which is adjusted to the target animal group, in order to create the moulded part.

5. If necessary: Addition of the protective layer, preferably centred on the top of the moulded part.

6. Compressing of the mass, in order to achieve the desired hardness, which is preferably adjusted to the target species.

7. Letting it dry. For that purpose the mould should be made of a suitable material, which allows the evaporation of the contained water. The hardening should preferably take place under slightly increased temperatures, while the product is under pressure or is still being compressed, for example in the air or in a drying oven.

In the case of application, the licking element can also be equipped with a support, which can be anchored in the ground. However, the licking element may also be installed on reusable supports naturally present at the place of installation.

The following describes a possible application on the basis of the following figures, which show in the form of a diagram:

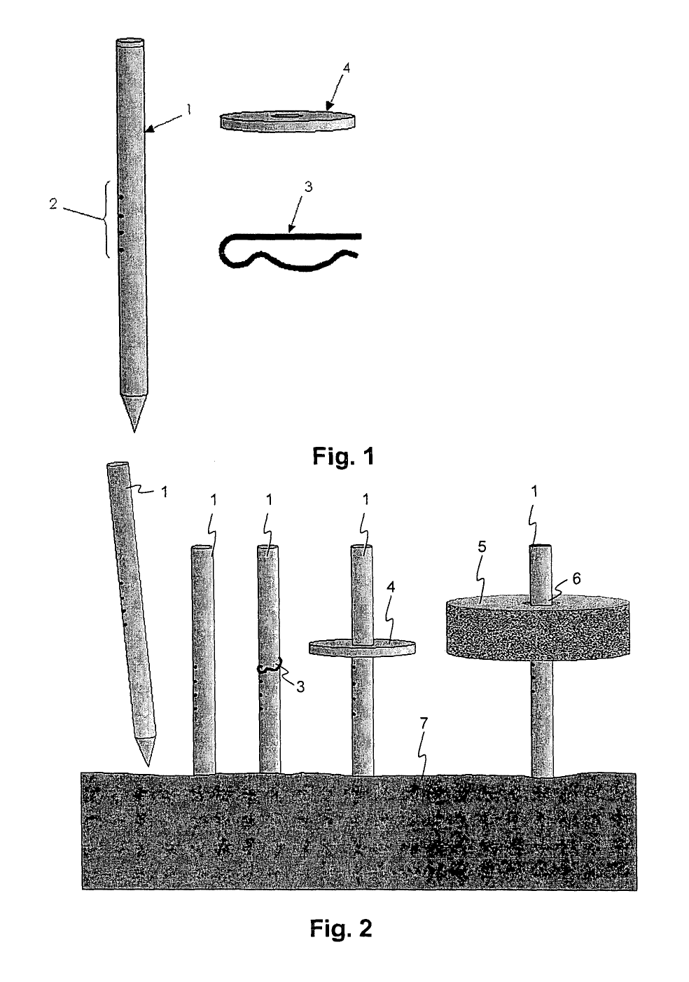

FIG. 1 The components of a support for a licking element

FIG. 2 Steps for the installation of the licking element at the place of installation.

The overall arrangement consists of a Licking Element 5 in the form of a cylindrical disk with a central Opening 6, a Support 1 (for example a metal pipe with a sharpened point), a Locking Element 3 (e.g. a pin), a Support Disk 4 and a protective cap that is integrated into the Licking Element 5 and which is not shown here. As FIG. 2 shows, the Support 1 is planted in the Ground 7 at the place of installation. Along its axis the Support 1 preferably has Holes 2, into which the Locking Element 3 can be inserted. In that manner the height of the Licking Element 5 above the ground can be adjusted. The Support Disk 4 is mounted on the Support 1 and held at the predetermined height by the Locking Element 3. The width of the Support Disk 4 is greater than the length of the Locking Element 3, so that the animals will not hurt themselves on the latter when licking. The Support Disk 4 should have a smaller diameter than the Licking Element 5 and should not have any sharp edges, which might hurt the animals. Afterwards the Licking Element 5 is placed on the Support Disk 4, with the side of the protective layer on top. The central Opening 6 of the Licking Element 5 is preferably adjusted to the form of the Support 1 in such way, that the Licking Element 5 cannot rotate during usage. If necessary, it is also possible to place several Licking Elements 5 on one Support 1, for example in order to avoid having to replace them frequently.

The steps for producing the licking element may be adapted to produce the compound in other forms by following steps 1 and 2 for producing the licking element. In broad terms, the process for producing the compound may comprise: mixing the binding agent with water to form a first mixture; adding the zeolite and the salt to the first mixture and stirring to form the compound. Specifically, the binding agent, for example rice bran, with 1-30% by weight of the target mass is mixed with de-ionised water and the mixture is then heated to approx. 60-80° C. The mixture is stirred until the bran starts to dissolve. Further, de-ionised water should be added while at the same time adding the remaining ingredients and mixing of the same, i.e. at least zeolite and salt, until the compound is formed.

The following describes tests, which illustrate the effectiveness of the claimed compound:

Test Series 1

The fresh contents of two rumens were mixed and distributed equally into two test containers (A, B). The mixing was done in order to compensate for the differences in digestion of the two slaughtered animals. Both containers contained 48 kg of rumen content. In a water basin the containers A+B were kept at a temperature of 38 degree Celsius, which simulates the body temperature of live animals. 60 grams zeolite of the type Clinoptilolite, corresponding to approx. 0.13% of the test matter, were added to container B. Afterwards the amount of gas escaping from the container was measured over a period of 10 hours using a gas flow measuring device type GWF, a one-pipe positive displacement gas meter.

The result, covering 4 test series, shows a reduction of CH₄ of 53 to 81% and of NH₄⁺ of 63% to 88%.

Test Series 2

Fresh contents of one fresh rumen was distributed equally into two test containers (A, B). Both containers contained 24 kg of rumen content. The reduced quantity allowed the continuous movement of the test containers during the test. In a water bath the containers A+B were kept at a temperature of 38 degree Celsius, which simulates the body temperature of live animals. 30 grams zeolite, corresponding to approx. 0.13% of the test matter, were added to container B. Afterwards the amount of gas escaping from the container was measured over a period of 10 hours using a gas flow measuring device type GWF, a one-pipe positive displacement gas meter.

The result, covering 4 test series, shows a reduction of CH₄ of 52 to 83% and of NH₄⁺ of 63% to 88%.

1. Compound for binding undesirable gases which are produced in digestive system of ruminants, comprising 50-90% by weight of zeolite, 1-20% by weight of salt as well as 1-15% by weight of a binding agent approved for foodstuffs.

2. Compound in accordance with claim 1, comprising 75-85% by weight of zeolite, 5-15% by weight of salt as well as 1-15% by weight of a binding agent approved for foodstuffs.

3. Compound in accordance with claim 1, wherein the zeolite has a grain size in the range of 10-150 μm.

4. Compound in accordance with claim 1, wherein the zeolite has a pore or channel size of 0.3 nm to 5 nm.

5. Compound in accordance with claim 1, wherein the zeolite includes a Clinoptilolite.

6. Compound in accordance with claim 1, wherein the binding agent includes at least one of the following substances: rye-, wheat-, rice- or other grain bran, E400 to E466, modified starch, in particular of the groups E1404-E1451, with the binding agent preferably being free of sweeteners.

7. Compound in accordance with claim 1, further comprising at least one of the following substances: minerals, pharmaceuticals, distilled oils, colouring.

8. Licking element for ruminants with at least one moulded part, which has a composition in accordance with claim 1.

9. Licking element in accordance with claim 8, further comprising a biodegradable protective layer, which is arranged on the top of the licking element, in particular a starch-based foil.

10. Licking element in accordance with claim 8, wherein the moulded part has the shape of a disk, a block or a rod.

11. Process for the production of a licking element in accordance with claim 8, characterised by the following steps:
Mixing the binding agent with water;
Adding the zeolite and the salt;
Stirring until a homogeneous mass has been achieved;
Filling of the mass into a mould;
Compressing;
Hardening, in order to produce the moulded part.

12. Process in accordance with claim 11, characterised by the addition of a protective layer prior to the step of compression.

13. Process in accordance with claim 11, characterised by the fixing of the hardened moulded part on a support.

14. Process in accordance with claim 11, wherein the zeolite is added in the form of a powder with a grain size of 10-150 μm.

15. Process for producing a compound according 7 claim 1, the process comprising:
Mixing the binding agent with water to form a first mixture;
Adding the zeolite and the salt to the first mixture and stirring to form the compound.
